(19) United States
(12) Patent Application Publication
(10) Pub. No.: US 2005/0214226 A1
(43) Pub. Date: Sep. 29, 2005

(54) Method of producing hollow, microporous particles which are intended, in particular to be inhaled

(76) Inventors: El Hassane Larhrib, Tourcoing (FR); Joseph Chukwuemeka Okpala, Liverpool (GB)

Correspondence Address: WADDEY & PATTERSON, 414 UNION STREET, SUITE 2020, BANK OF AMERICA PLAZA, NASHVILLE, TN, 37219, US

(21) Appl. No.: 10/501,485

(22) PCT Filed: Jan. 14, 2003
(86) PCT No.: PCT/FR03/00102

§ 371 Date: May 6, 2005

(30) Foreign Application Priority Data
Jan. 15, 2002 (FR) 02/00427

(51) Int. Cl. A61L 9/04; A61K 9/14

(52) U.S. Cl. 424/46; 264/5

(57) ABSTRACT

The invention relates to a method of producing hollow, microporous particles which are intended, in particular, to be inhaled or for any other application. The inventive method consists in: providing a composition, in a given form, comprising at least one active principle and at least one blowing agent; cooling the aforementioned composition to below the solidification point of said at least one blowing agent so as to increase the volume of the given form and to create ruptures in the surface and/or in all of said given form, thereby enabling the structure of the hollow, microporous particle to be obtained; and removing all or part of said at least one blowing agent.

The present invention relates to a method of producing hollow microporous particles which are intended in particular to be inhaled as well as to said hollow microporous particles produced by the method.

Although the present invention is more specifically designed to produce hollow microporous particles intended to be inhaled, it is in no way limited to such an application; in contrast thereto, the present invention is applicable in all areas in which it is advantageous to use such hollow microporous particles no matter how they are administered or used.

Additionally, the invention relates to an inhaler device comprising the hollow microporous particles produced by the method.

Powders containing particles to be inhaled by a patient have been known in the pharmaceutical industry for a number of years. These powders formed of said particles contain different substances such as active principles, intended to treat different types of illnesses such as respiratory diseases.

Generally, in this case, inhalation of the particles should enable them to be deposited in the bronchial tubes and/or lungs. That being said, taking into account the nature of the particles hitherto known, they were either too dense and were deposited before the correct depositing zone, for example in the mouth or in the throat or even in the inhaler device, or light particles were used, which enable the zone to be treated to be reached but not without creating difficulties in terms of flow which are inherent to their structural characteristics and to inter-particulate cohesion forces.

The concept of hollow microporous particles has been introduced to circumvent the above drawbacks. These particles have, in fact, a double advantage: on the one hand they have a low density permitting their inspiration and the diffusion of the micro-particles in the regions far from the mouth to be facilitated, and on the other hand they have a high surface area permitting better reactivity of the particles with the surface to be treated, as well as a better flow.

These known hollow microporous particles are generally prepared from a mixture of active principles and an agent permitting rapid freezing, said mixture being sprayed in the form of droplets onto a cold medium. The frozen droplets are then freeze-dried and dried permitting the solvent to be removed and thereby creating the microporous particles.

That being said, such known production methods have different drawbacks, and in particular require two successive steps to be implemented including an unavoidable long and costly freeze-drying step. Furthermore, the method above does not always permit certain limitations in terms of the density and surface area of the particles to be overcome in a simple and economic manner.

Moreover, these methods require steps which are difficult to implement, the porosity of the particles actually being obtained by subliming the solvent during the freeze-drying rapid freezing/drying method.

The object of the present invention is to propose a method of producing hollow microporous particles, which are intended in particular to be inhaled or any other application, which method overcomes the aforementioned drawbacks and enables the production of hollow microporous particles having very low density and high specific surface area.

Another object of the present invention is to propose a simplified method of producing hollow microporous particles, which are intended in particular to be inhaled or any other application, which method enables good control of the physical characteristics of the hollow microporous particles and which is economic to implement and enables simple industrial transposition.

Another object of the present invention is to propose a medicine intended to be administered by inhalation, particularly for the treatment of respiratory diseases, as well as a corresponding inhaler device which are particularly suitable and efficient. Furthermore, in accordance with the invention, suitable particles composed of up to 100% of the active product may be provided.

Other objects and advantages of the present invention will become apparent from the following description which is given by way of example only and does not limit the invention.

In accordance with the invention, the method of producing hollow microporous particles intended in particular to be inhaled or any other application, is characterised in that:
a composition in a given form, comprising at least one active principle and at least one expansion agent, is sprayed,
said composition is cooled at a temperature below the solidification point of said at least one expansion agent so as to increase the volume of the given form and to create fractures in the surface and/or in all of said given form, thereby enabling the structure of a hollow microporous particle to be obtained.
all or part of said at least one expansion agent is removed.

Furthermore, the hollow microporous particles produced in accordance with the present invention include particles measuring from 0.1 μm up to 2000 μm and having a powder density between 0.4 g/cm³ and 0.000 g/cm³.

Moreover, according to the present invention, said micro-particles are used to produce a medicine intended to be administered by inhalation, in particular for the treatment of respiratory diseases. However, it should be noted that it may be administered by any conventional method.

The invention will be better understood upon reading the following description, accompanied by the appended figures which form an integral part of the description and in which:

FIG. 1 is a view of a first type of 100% active product, in an initial micronised form, implemented in the method of the present invention,

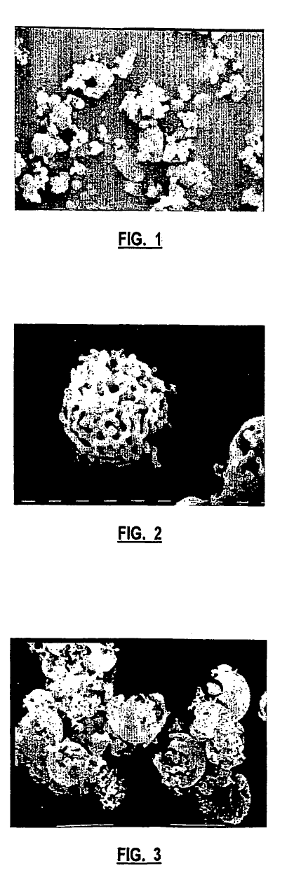

FIG. 2 represents an example of hollow microporous particles, in accordance with the invention, made from said micronised 100% active product illustrated in FIG. 1, in accordance with a first mode of operation,

FIG. 3 represents a second example of hollow microporous particles, in accordance with the invention, obtained from said micronised 100% active product illustrated in FIG. 1, in accordance with a second mode of operation,

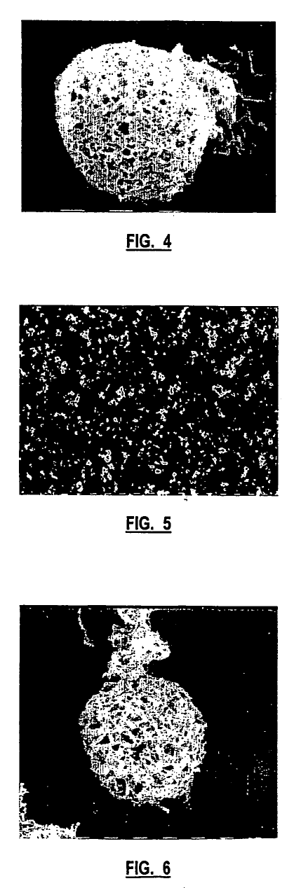

FIG. 4 represents a third example of hollow microporous particles, in accordance with the invention, obtained from the micronised active product illustrated in FIG. 1, combined with an excipient, in accordance with said first mode of operation,

FIG. 5 represents a view of another 100% active product in an initial micronised form, implemented by the method of the present invention,

FIG. 6 represents a view of the hollow microporous particles obtained from said other active product represented in FIG. 4, combined with an excipient.

The present invention relates to a method of producing hollow microporous particles such as those illustrated in FIGS. 2, 3, 4 or 6, in particular intended to be inhaled, wherein a composition is provided in a given form, such as that illustrated in FIGS. 1 or 5, comprising at least one active principle and at least on expansion agent.

In this regard, the term “active principle” is intended to mean any product or substance of given characteristics having the specific desired effect.

In one embodiment of the invention, said composition is sprayed in particular by atomisation in the form of droplets having given dimensional characteristics.

This atomisation can be carried out by any method known by the person skilled in the art, and in particular using pneumatic means, ultrasonic means, pressurized means, nozzle means, rotary atomiser means, blowing means, high rotational generators, spraying devices, gauge needles or a hair-dryer.

With particular reference to FIGS. 2 and 3, it can be seen that the atomising distance, for a single said composition, modifies the final structure of the hollow micro-porous particle obtained.

For FIG. 2, atomisation was effected at about 70 cm from the intermediate cooling medium, described in more detail below, whereas for FIG. 3 the atomising distance was only 10 cm.

It is important to note at this point that the small white bars at the bottom of each picture of FIGS. 1, 2, 4, 5 and 6 correspond to a distance of 1 μm, and 10 μm for FIG. 3.

Other atomising parameters are equally important, by way of example, increasing the atomisation pressure leads to the formation of smaller droplets, the same applies to the flow of atomised liquid.

The atomisation gas is advantageously selected from the group consisting in particular of carbon dioxide, nitrogen, argon, oxygen, air and combinations thereof. That being said, other gases, in particular inert gases, may also be envisaged.

As mentioned above, said composition comprises at least one active principle and at least one expansion agent. Said at least one active principle is intended, for example, for therapeutic, prophylactic or even diagnostic use. There is clearly a large number of active principles which can be used by inhalation.

That being said, certain active principles are better adapted to this type of use and amongst them, by way of example, the active principles selected from the group consisting of proteins, lipids, nucleic acid, short-chain peptides, corticosteroids, anti-inflammatory medicines, analgesics, neoplastic agents, antitussives, bronchodilators, diuretics, anticholinergics, hormones, anginal preparations, antiallergics, anti-infectives, anti-histamines, anti-tuberculous agents, therapeutic proteins and peptides.

More precisely, as the active principle in anti-infalmmatories, the following compounds could also be used: beclomethasone, betamethasone, fluticasone, flunisolide, budesonide, dexamethasone, tipredane, triamcinolone acetonide.

The antitussive could include in particular the compound noscarpine.

The bronchodilators could include the compounds: ephedrine, adrenaline, fenoterol, formoterol, isoprenaline, metaproterenol, phenylephrine, phenylpropanolamine, pirbuterol, reproterol, rimiterol, salbutamol salmeterol, formoterol, terbutaline, isoetharine, tulobuterol, orciprenaline or (−)-4amino-3,5-dichloro-α[[[6-[2-(pyridinyl)ethoxy}hexyl]amino]methyl]benzenemethanol.

Amiloride could in particular be used as a diuretic. The anticholinergics could include the following compounds: ipratropium, ipatropium bromide, atropine, oxitropium or oxitropium bromide.

The hormones could include in particular cortisone, hydrocortisone or prednisolone.

The xanthines could include in particular: aminophylline, choline theophyllinate, lysine theophyllinate or theophylline.

The analgesics could include in particular the following compounds: codeines, dihydromorphine, ergotamine, fentanyl or morphine.

The anginal preparations could include diltiazem.

The antiallergics could include cromoglycate, ketotifen or nedocromyl.

The anti-infectives could include the following compounds: cephalosporin, penicillin, streptomycin, sulphonamides, tetracyclines or pentamidines.

The anti-histamines could include in particular methapyrilene.

The anti-neoplastic agents could include bleomycin, carboplatin, methotrexate, adriamycin, amphotericin B.

The anti-tuberculous agents could include in particular isoniazide or ethanbutol.

Finally, the therapeutic proteins and peptides could include insulin, glucagon, prostaglandin, leukotrienes as well as their activators and inhibitors including prostacyclin (epoprostanol), and prostaglandins E₁ and E₂.

Generally, any other type of agent which can be delivered in particular via inhalation can be used for prophylactic, therapeutic, or diagnostic purposes.

Furthermore, said active principles could be used in the form of salts such as alkali metal, acid addition salt or in the form of ester such as lower alkyl ester or in the form of solvate such as hydrates so as to optimise the activity and/or stability of said active principles. In accordance with a preferred embodiment, said active principle is selected from the group consisting of anti-inflammatories or bronchodilators. More particularly, the preferred active principles are beclomethasone dipropionate and salbutamol sulphate.

In another embodiment of the invention, nutrients could also be used and these include in particular, by way of example, retinoids such as all-cis retinoic acid, 13-trans retinoic acid and others, vitamins A as well as beta carotene derivatives, vitamins D, E, K, water insoluble precursors as well as derivatives thereof

Said at least one expansion agent is formed from a liquid or gas whose volume after cooling below the solidification point is greater than the volume in the liquid or gaseous state. It is also preferred that solvents having high volatile properties in addition to an expansion co-efficient are used as the expansion agent.

In fact, upon projection, it is interesting to note that the expansion agent is volatile so as to evaporate from the surface of the droplets during said projection.

Furthermore, numerous solvents can be used as expansion agents, including by way of non-limiting example, chlorofluorocarbons such as perfluorocarbons such as perfluorooctyl bromides, freon, exotic solvents such as hexafluoroisopropanol, hexafluorides, hexafluorocyclobutanes, fluorocarbon refrigerants such as dichlorodifluoromethane, perfluoropropane, CF4,C2F6,C3F8, C4F8, C2F4, C3F6, inhalation anaesthetics such as halothane, enflurane, isoflurane, methoxyflurane, sevoflurane, hydrofluoroalkane such as HFA-134a, HFA-227, ketones such as acetones, alcohols such as ethanol, tertiary butyl alcohols, methanol and other organic solvents such as dichloromethanes, chloroforms, acetonitrile, dioxane, dimethylsulfoxide, ethyl acetate, methyl acetate, tetrahydrofurane (THF). Volatile salts such as ammonium bicarbonate, ammonium acetate, ammonium chloride, ammonium benzoate. Preferably, the expansion agents used have a low toxicity and are pharmaceutically accessible.

That being said, in accordance with an advantageous embodiment giving good results, acetone is used as the expansion agent.

Similarly, a corticosteriod such as beclomethasone dipropionate (BDP), as illustrated in FIGS. 2, 3 and 4, could be used as the active principle in the presence of cellulose acetate phthalate (CAP), as illustrated in FIG. 4, if need be.

In an alternative embodiment, good results were obtained when using a bronchodilator, such as salbutamol sulphate, as the active principle, in the presence of CAP if need be, as illustrated in FIG. 6.

Furthermore, gasses could also be used as expansion agents; these gasses including the gas group chosen from dissolved gases such as carbon dioxide, nitrogen, from gas-producing agents such as carbonate, bicarbonate, carboxylic acid as well as derivatives thereof or from nitrogen-producing agents.

It is also possible to use said expansion agents in the liquid or gaseous form in combination.

It should be noted here that in accordance with another embodiment a composition could be provided in a given form formed from microparticles including a residual expansion agent.

That being said, the method of producing hollow microporous particles consists in, after spraying in particular said composition, cooling said composition below the solidification point of said at least one expansion agent, in order to increase the volume of the given form and to create fractures on the surface and/or on the whole of said given form permitting the structure of the hollow microporous particle to be obtained.

To do so, the composition was sprayed onto a cold medium having a temperature below said solidification point of said at least one expansion agent. Cooling is carried out in particular by freezing using a gas advantageously selected from the group consisting of liquid hydrogen, liquid nitrogen, liquid argon and liquid oxygen.

Thus, taking the example of a composition comprising acetone as the expansion agent whose solidification point is at a temperature of −95.4° C., it is possible to use liquid nitrogen gas at a temperature of −185.6° C. The expansion coefficient of acetone is approximately 108%, the expansion coefficient being calculated using the following formula;
expansion %=[(V_{F}−Vᵢ)/Vᵢ]×100
where Vᵢ represents the initial volume and V_{F} the final volume of the expansion agent.

It is equally possible to use liquid nitrogen gasses with other expansion agents and in particular dichloromethane which has an expansion coefficient of about 20% and a solidification point of −95.1° C., methanol which has an expansion coefficient of about 36% and a solidification point of −97.5° C. or even carbonated water which has an expansion coefficient of about 33% and a solidification point of 0° C.

In order to be able to increase the volume of said given form sufficiently and in order to be able to create fractures in the surface and/or in the whole of said form, it is necessary for the expansion agent to have a minimum expansion coefficient. Thus, by way of example, Water cannot be considered as an expansion agent, since the expansion co-efficient of water at close to 2% is in fact relatively small and insufficient.

It has been shown that it is advantageous to use an expansion agent whose expansion rate is greater than 5% in order to obtain a good structure of hollow microporous particles.

It should also be noted that the very low temperature obtained by freezing weakens the resistance of the structure of said active principle rendering it more brittle, hence, the surface and/or the whole of the latter becomes subject to fracturing taking into account the stress imparted by the expansion of the expansion agent, from the inside towards the outside of the particle or vice versa.

That being said, it could also be envisaged to add other solvents, such as water, to said expansion agent and to said at least one active principle. Thus, the composition can comprise, for example, a mixture of acetone and water in a ratio of 80 to 20 volume/volume.

It is also possible to use at least one additional excipient in the composition. Said additional excipient could be intended in particular to permit the density to be altered, the action of said at least one active principle to be slowed, controlled or targeted, the excipient could be for example a polymer compound.

It is important to note at this point that the active principle can be combined in different ways with said at least one expansion agent and optionally with said at least one additional excipient. Thus, the active principle can be dissolved, emulsified or suspended, alone or in combination, in said at least one expansion agent and if need be with said at least one additional excipient.

Furthermore, it can be envisaged to combine one or more hydrophobic or hydrophilic active principles, indeed to provide them in combination with other excipients. Thus, in a preferred embodiment, the composition comprises a short acting Beta 2 agonist in combination with an anti-muscarinic agent such as salbutamol in combination with ipatropium bromide; or feneterol in combination with ipatropium bromide. Another preferred composition could comprise a short-acting Beta 2 agonist in combination with a corticosteroid, such as salbutamol and beclomethasone. Another preferred composition could comprise a long-acting Beta 2 agonist in combination with a corticosteroid such as salmeterol and fluticasone or formeterol and budesonide.

In this regard, the term “hydrophobic” is intended to mean a substance which is insoluble, or only slightly soluble in water. In the case of the present invention, it relates to active principles and/or excipients which can have a solubility below 10 mg/ml, indeed below 1 mg/ml and even below 0.01 mg/ml.

Furthermore, the term “hydrophilic” is intended to mean a substance which is highly soluble in water and/or is capable of swelling and forming a gel. In the case of the present invention, it relates to active principles and/or excipients which can have a sensitivity in aqueous medium greater than 5 mg/ml, indeed greater than 50 mg/ml and even greater than 100 mg/ml or more.

Several excipients can be used and in particular by way of non-limiting example the excipient can be a non-biodegradable, biodegradable or bioerodible polymer, i.e., a polymer which enzymatically or chemically degrades in vivo into non toxic small molecules.

Suitable polymers for the applications mentioned can be synthetic or natural and can include in particular cyclodextrins and derivatives thereof, sodium caseinate, DPPC, human serum albumin, cellulose acetate phthalate, phospholipids, hydroxypropyl methylcellulose phthalate, ethyl cellulose, hydroxypropyl methyl cellulose, hydroxyethyl cellulose, hydroxypropyl cellulose, hydroxyethyl cellulose, carboxymethyl cellulose, methyl cellulose, cellulose acetate butyrate, poloxamer, poly(lactic acid), poly(lactic glycolic acid), poly(lactide), poly(glycolide), poly(lactide-coglycolide), poly(p-dioxanone), poly(caprolactone), polycarbonate, polyamide, polyanhydride, poly(alkylene alkylate), polyamino acid, polyhydroxyalkanoates, polypropylenefumarates, polyorthoester, polyacetal, polyacrylamide, polycyanoacrylate, polyalkylcyanoacrylates, polymethapolyphosphate ester, polyphosphazene, polyurethane, polyacrylate, polymethacrylate, poly(methyl methacrylate), poly(hydroxy ethyl methacrylate-co methyl methacrylate), carbopol 934, ethylene-vinyl acetate and other substituted acyl cellulose acetates and derivatives thereof, polystyrene, polyvinyl alcohol, polyvinyl pyrrolidone, polyvinyl chloride, polyvinyl fluoride, poly(vinyl imidazole), chlorosulphonated polyolefin, polyethylene, polyethylene glycol, polypropylene, polyethylene oxide, copolymer and blends thereof. Preferably, the selected polymer is biocompatible and degrades or erodes in vivo to form small non-toxic molecules. More preferably, the polymer is biocompatible and pharmaceutically acceptable for delivery to the respiratory tract. Finally, the polymer, in addition to being pharmaceutically acceptable, can have therapeutic properties. A particularly suited polymer could include in particular cellulose acetate phthalate (CAP) or hydroxypropyl cellulose acetate phthalate, polymeric medicines or genetically-engineered polymers.

The process of producing hollow microporous particles further consists, if required, of removing whole or part of said at least one expansion agent. This removal is carried out in particular by evaporation through pores created by the fractures on the surface of said given form of increased volume. Hollow microporous particles, only containing a residual amount of expansion agent or even no expansion agent, are thus obtained as shown in FIGS. 2, 3, 4 or 6.

In this regard, the production method can further comprise an additional step wherein said obtained hollow microporous particles are dried. The drying step can be carried out by any technique known to those skilled in the art in the field of drying and in particular can be carried out using a conventional oven, vacuum oven, fluid bed dryer or blowing means.

In accordance with an advantageous embodiment of the invention, said drying step comprises a step of evaporating said at least one expansion agent, permitting the expansion agent residues to be evaporated, which residues were not removed during the evaporation step through the pores of the structure and during the previous step.

In accordance with another embodiment of the invention, the drying step may also comprise a step of lyophilising said hollow microporous particles.

In one embodiment of the invention, the obtained hollow microporous particles have a diameter of between 0.1 μm and 2000 μm, and advantageously between 0.1 μm and 100 μm, the density of the corresponding particles is less than 0.5 g/cm³ and as low as 0.0001 g/cm³.

Several examples which the invention can produce can be seen in the following table, in relation to the accompanying Figures:

| | Mean | | | Angle |
|---|---|---|---|---|
| | volumetric | | Specific | of |
| | diameter VMD | Density | surface area | slide |
| Particle | (μm) | (g/cm³) | m²/g | (θs) |
| | 3.17 ± 0.38 | 0.168 ± 0.002 | 3.538 ± 0.01 | 70.00 ± |
| | | | | 5.00 |
| | 8.83 ± 1.48 | 0.031 ± 0.000 | 7.070 ± 0.00 | 43.67 ± |
| | | | | 2.52 |
| | 7.06 ± 1.02 | 0.0107 ± 0.002 | 21.75 ± 0.01 | 46.17 ± |
| | | | | 3.27 |
| | 4.05 ± 0.03 | 0.233 ± 0.011 | 4.255 ± 0.010 | 60.00 ± |
| | | | | 6.25 |
| | 6.02 ± 1.12 | 0.0025 ± 0.001 | 54.050 ± 0.015 | 42.17 ± |
| | | | | 3.75 |

As indicated previously, said hollow microporous particles could be used in the production of a medicine, in particular for the treatment of respiratory disorders.

To this end, said hollow microporous particles obtained by the method as set out herein will be administered using an inhaler device comprising said hollow microporous particles.

Of course, other embodiments within the scope of those skilled in the art could be envisaged without departing form the scope of the invention as defined in the Claims hereinafter. In this regard, it is clear that said hollow microporous particles could be used alone as they are or also in the presence of diluents. They may be used as they are, fragmented or ground.

The diluents are used to improve the dispersion of the powder in the inhaler device and/or to improve the flow and manageability of the powder. The diluents could include in particular monsosaccharides such arobinose, xylitol and dextrose and monohydrates, either di-saccharides such as lactose, maltose and sucrose, or poly-saccharides such as starch, dextrins or dextrans. Advantageously, lactose monohydrate could be chosen as the diluant, the amount of which to be added to the microporous particles of the present invention will adjusted by the person skilled in the art, so that for example the final amount of the composition is from 0.1 to 90% w/w.

1. Method of producing hollow microporous particles in particular intended to be inhaled or any other application characterised in that:
a composition is provided in a given form comprising at least one active principle and at least one expansion agent,
said composition is cooled at a temperature below the solidification point of said at least one expansion agent so as to increase the volume of the given form and to create fractures in the surface and/or in all of said given form, thereby enabling the structure of the hollow micro-porous particle to be obtained,
all or part of said at least one expansion agent is removed.

2. Method as claimed in claim 1, wherein said composition in a given form is sprayed onto a cold medium having a temperature lower than said solidification point of said at least one expansion agent.

3. Method as claimed in claim 1, wherein said at least one expansion agent has an expansion coefficient greater than 5%.

4. Method as claimed in claims 1 to 3, wherein said at least one expansion agent is selected from the group consisting of methanol, dichloromethane, acetone, and mixtures thereof.

5. Method as claimed in claims 1 to 3, wherein said at least one expansion agent is selected from the group of gas consisting of carbon dioxide, nitrogen, carbonate, bicarbonate, carboxylic acid and derivatives thereof.

6. Method as claimed in any one of the preceding Claims, wherein said active principle is selected from the group consisting of proteins, lipids, nucleic acid, short chain peptide, corticosteriods, anti-inflammatories, analgesics, anti-neoplastic agents or bronchodilators.

7. Method as claimed in any one of the preceding Claims, wherein said active principle is a steroid selected from the group consisting of budesonide, testosterone, progesterone, oestrogen, flunisolide, triamcinolone, beclomethasone, betamethasone, dexamethasone, fluticasone, methyl-prednisolone, prednisone and hydrocortisone.

8. Method as claimed in one of claim 7, wherein the active principle is beclomethasone dipropionate (BDP).

9. Method as claimed in claim 6, wherein said active principle is a bronchodilator selected from the compounds ephedrine, adrenaline, fenoterol, formoterol, isoprenaline, metaproterenol, phenylephrine, phenylpropanolamine, perbuterol, reproterol, rimeterol, salbutamol, salmeterol, formorterol, terbutaline, isoetharine, tulobuterol, orciprenaline or (−)-4-amino-3,5-dichloro-α[[[6-[2-(pyridinyl)ethoxy}hexyl]amino]methyl]benzenemethanol.

10. Method as claimed in claim 9, wherein said active principle is salbutamol sulphate.

11. Method as claimed in claim 2, wherein the spraying step is carried out by atomising said composition in the form of droplets.

12. Method as claimed in the preceding Claim, wherein atomisation is carried out using pneumatic means, ultrasonic means, pressurized means, nozzle means, rotary atomiser means, blowing means, high rotational generators, spraying devices, gauge needles or a hair-dryer.

13. Method as claimed in the preceding Claim, wherein the atomisation gas is selected from the group consisting of carbon dioxide, nitrogen, argon, oxygen, air and mixtures thereof.

14. Method as claimed in any one of the preceding Claims, wherein the cooling step is carried out by means of a gas selected from the group consisting of liquid hydrogen, liquid nitrogen, liquid oxygen.

15. Method as claimed in any one of the preceding claims, wherein furthermore said particles are dried using blowing means, oven, vacuum oven, fluid bed dryer.

16. Method as claimed in claim 15, wherein the drying step comprises the evaporation of said at least one expansion agent.

17. Method as claimed in claim 15, wherein the drying step comprises the lyophilisation of the particles.

18. Method as claimed in any one of the preceding Claims, wherein the composition comprises a mixture of acetone and water in a ratio of 80:20 volume/volume.

19. Method as claimed in any one of the preceding Claims, wherein the composition further comprises at least one additional excipient.

20. Method as claimed in claim 19, wherein said at least one additional excipient is a polymer compound permitting the density to be altered and the action of said at least one active principle to be slowed, controlled or targeted.

21. Method as claimed in claim 19, wherein said at least one additional excipient is selected from the following compounds: cyclodextrins and derivatives thereof, sodium caseinate, DPPC, serum albumin, cellulose acetate phthalate, phospholipids, hydroxypropyl methylcellulose phthalate, ethyl cellulose, hydroxypropyl methyl cellulose, hydroxyethyl cellulose, hydroxypropyl cellulose, hydroxyethyl cellulose, carboxymethyl cellulose, methyl cellulose, cellulose acetate butyrate, poloxamer, poly(lactic acid), poly(lactic glycolic acid), poly(lactide), poly(glycolide), poly(lactide-coglycolide), poly(p-dioxanone), poly(caprolactone), polycarbon, polyamide, polyanhydride, poly(alkylene alkylate), polyamino acid, polyhydroxyalkanoates, polypropylenefumarates, polyorthoester, polyacetal, polyacrylamide, polycyanoacrylate, polyalkylcyanoacrylates, polymethapolyphosphate ester, polyphosphazene, polyurethane, polyacrylate, polymethacrylate, poly(methyl methacrylate), poly(hydroxy ethyl methacrylate-co methyl methacrylate, carbopol 934, ethylene-vinyl acetate and other substituted acyl cellulose acetates and derivatives thereof, polystyrene, polyvinyl alcohol, polyvinyl pyrrolidone, polyvinyl chloride, polyvinyl fluoride, poly(vinyl imidazole), chlorosulphonated polyolefin, polyethylene, polyethylene glycol, polypropylene, polyethylene oxide, copolymer and blends thereof, cellulose acetate phthalate (CAP) and hydroxypropyl cellulose acetate phthalate.

22. Hollow microporous particles produced by the method as claimed in any one of claims 1 to 21, having particles measuring between 0.1 μm and 2000 μm and whose density is in the range from 0.4 g/cm³ to 0.0001 g/cm³.

23. Medicine, intended to be administered by inhalation, having the microporous particles as claimed in claim 22.

24. Use of said hollow microporous particles as claimed in claim 22 in the production of a medicine for treating respiratory diseases.

25. Inhalation device having the hollow microporous particles obtained by the method as claimed in any one of claims 1 to 21.
